(19) United States
(12) Patent Application Publication
(10) Pub. No.: US 2025/0169694 A1
(43) Pub. Date: May 29, 2025

(54) RETINAL IMAGING FOR DETECTION OF BIOMARKERS ASSOCIATED WITH NEURODEGENERATIVE DISEASES

(71) Applicant: Welch Allyn, Inc., Skaneateles Falls, NY (US)

(72) Inventors: Allen R. Hart, Maryville, TN (US); Hongying Krause, Cranleigh (GB)

(21) Appl. No.: 18/935,929

(22) Filed: Nov. 4, 2024

Related U.S. Application Data
(60) Provisional application No. 63/604,030, filed on Nov. 29, 2023.

(51) Int. Cl. A61B 3/12; A61B 3/14; A61B 5/00; G16H 30/40; G16H 50/20

(52) U.S. Cl. CPC A61B 3/12; A61B 3/14; A61B 5/4088; A61B 5/7275; G16H 30/40; G16H 50/20

(57) ABSTRACT

A device for neurodegenerative disease screening is described. The device operates an imaging assembly to capture a retinal image using an illumination path that is offset with respect to an optical path. The device receives a recommendation based on detection of a biomarker in the retinal image. The biomarker is associated with a neurodegenerative disease.

### CROSS REFERENCE TO RELATED APPLICATION

This application claims priority to U.S. Provisional Patent Application No. 63/604,030, filed Nov. 29, 2023, the disclosure of which is hereby incorporated by reference in its entirety.

### BACKGROUND

Alzheimer's is a progressive disease that destroys memory and other important mental functions. Typically, Alzheimer's causes brain cell connections and the cells themselves degenerate and die, eventually destroying memory and other important mental functions. Memory loss and confusion are the main symptoms. Currently, there is no cure for Alzheimer's. However, there are medications and management strategies that may slow down the progression of the disease. Thus, early detection of Alzheimer's symptoms is advantageous.

### SUMMARY

In general terms, the present disclosure relates to retinal imaging for detection of a biomarker associated with a neurodegenerative disease. In one possible configuration, an eye imager generates an off-axis illumination of the retina for capturing a retinal image that is analyzed for detection of a biomarker associated with Alzheimer's disease. Various aspects are described in this disclosure, which include, but are not limited to, the following aspects.

One aspect relates to a device for neurodegenerative disease screening, the devise comprising: an imaging assembly including: an image sensor for capturing a retinal image; a lens unit focusing light reflection from an eye toward the image sensor, the lens unit at least partially defining an optical path for receiving the light reflection; and an illumination unit having one or more light-emitting diodes emitting light in an illumination path for illuminating the eye, the illumination unit being mounted relative to the lens unit such that the illumination path is offset with respect to the optical path; at least one processing device; and at least one computer readable data storage device storing software instructions that, when executed by the at least one processing device, cause the device to: operate the imaging assembly to capture the retinal image using the illumination path offset with respect to the optical path; and receive a recommendation based on detection of a biomarker in the retinal image, the biomarker being associated with a neurodegenerative disease.

Another aspect relates to a method of screening for a neurodegenerative disease, the method comprising: capturing a retinal image by operating an imaging assembly having an illumination path that is offset with respect to an optical path; detecting a biomarker in the retinal image, the biomarker being associated with the neurodegenerative disease; and generating a recommendation based on detection of the biomarker.

Another aspect relates to a system for neurodegenerative disease screening, the system comprising: at least one processing device; and at least one computer readable data storage device storing software instructions that, when executed by the at least one processing device, cause the device to: receive a retinal image captured using a non-mydriatic workflow; screen the retinal image for amyloid plaque associated with Alzheimer's disease; screen the retinal image for conditions associated with eye diseases; and generate a recommendation, wherein the recommendation includes a referral to a neurologists when the amyloid plaque are detected in the retinal image, and wherein the recommendation includes a referral to an ophthalmologist when the conditions associated with the eye diseases are detected in the retinal image.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of an example of an eye imager that captures retinal images for detection of biomarkers associated neurodegenerative diseases.

FIG. 2 is a rear isometric view of the eye imager of FIG. 1.

FIG. 3 is a side view of the eye imager of FIG. 1 during an eye examination performed on a patient.

FIG. 4 schematically illustrates an example of the eye imager of FIG. 1.

FIG. 5 is a side view illustrating an example of an imaging assembly positioned relative to an eye of the patient such as during image capture by the eye imager of FIG. 1.

FIG. 6 schematically illustrates an example of a method of performing a retinal exam of the patient that can be performed by the eye imager.

FIG. 7 is an example of a retinal image captured by the eye imager of FIG. 1, the retinal image including biomarkers associated with a neurodegenerative disease.

FIG. 8 is another example of a retinal image captured by the eye imager of FIG. 1, the retinal image including biomarkers associated with a neurodegenerative disease.

FIG. 9 is another example of a retinal image captured by the eye imager of FIG. 1, the retinal image including biomarkers associated with a neurodegenerative disease.

FIG. 10 is another example of a retinal image captured by the eye imager of FIG. 1, the retinal image including biomarkers associated with a neurodegenerative disease.

FIG. 11 is another example of a retinal image captured by the eye imager of FIG. 1, the retinal image including biomarkers associated with a neurodegenerative disease.

FIG. 12 illustrates an example of a graphical user interface displayed in accordance with the method of FIG. 6.

FIG. 13 schematically illustrates an example of a computing device of the eye imager of FIG. 1.

### DETAILED DESCRIPTION

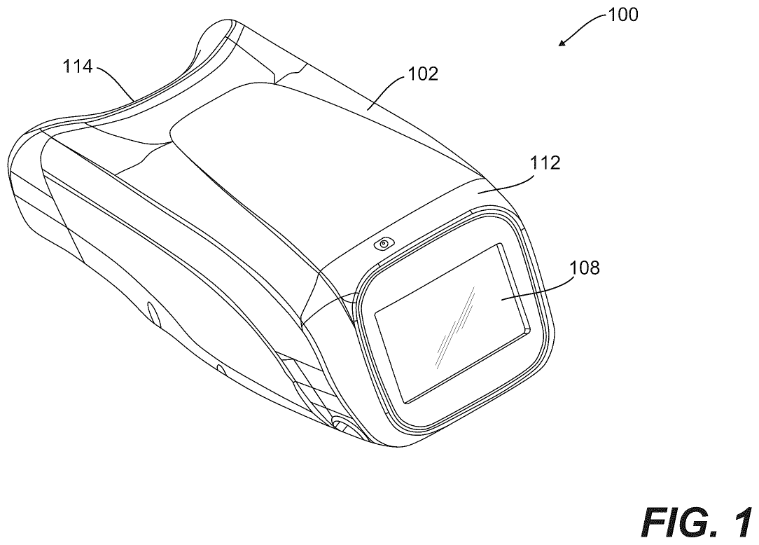

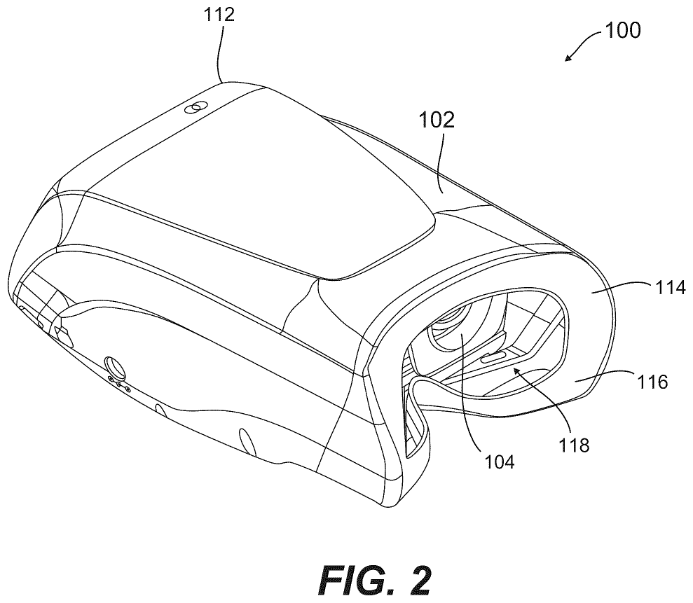

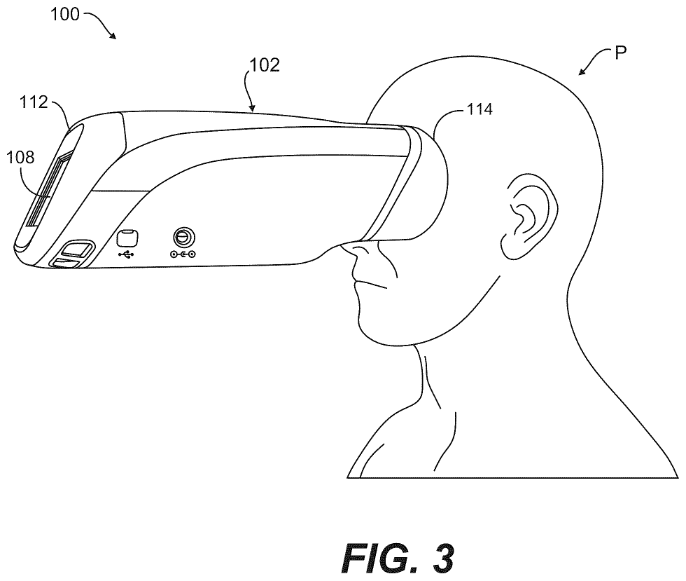

FIG. 1 is a front isometric view of an example of an eye imager 100 that captures retinal images for detection of biomarkers associated neurodegenerative diseases. FIG. 2 is a rear isometric view of the eye imager 100. FIG. 3 is a side view of the eye imager 100 during an eye examination performed on a patient P. The eye imager 100 is operable to capture and view retinal images of the patient P. The eye imager 100 can be used to screen, diagnose, and/or monitor progression of one or more eye diseases, such as retinopathy, macular degeneration, glaucoma, papilledema, and the like. The eye imager 100 can also be used to screen, diagnose, and/or monitor progression of one or more neurodegenerative diseases such as Alzheimer's.

The eye imager 100 is operable to screen for eye diseases and/or neurodegenerative diseases by capturing one or more retinal images, screening for the presence of biomarkers in the one or more retinal images, and providing a recommendation based on whether the biomarkers are present or not in the retinal images. The eye imager 100 can help users who are not trained specialists to screen for one or more eye diseases and/or one or more neurodegenerative diseases in a single exam setting by providing a recommendation to follow up with a specialist such as an ophthalmologist or neurologist based on detection of the biomarkers in the retinal images.

One technique for eye imaging requires mydriasis, or the dilation of the patient P's pupil, which can be painful and/or inconvenient to the patient P. The eye imager 100 does not require a mydriatic drug to be administered to the patient P before imaging, although the eye imager 100 can image the retina of the patient P when a mydriatic drug is administered.

As shown in FIGS. 1-3, the eye imager 100 includes a housing 102. The housing 102 supports a display 108 at a first end 112. The housing 102 further includes a second end 114 that is shaped and sized to engage the patient P's face (see FIGS. 2 and 3). The housing 102 of the eye imager 100 is sized and shaped to be handheld and portable.

The display 108 displays retinal images of the left and right eyes of the patient P. Additionally, the display 108 is also configured to display workflows and controls for capturing the retinal images in examples where the display 108 is a touchscreen. The housing 102 can additionally support one or more user input buttons near display 108. The display 108 can be used to initiate the image capture sequence, as described herein. Thus, the eye imager 100 can perform automatic and/or manual workflows for capturing retinal images of the patient P's eyes.

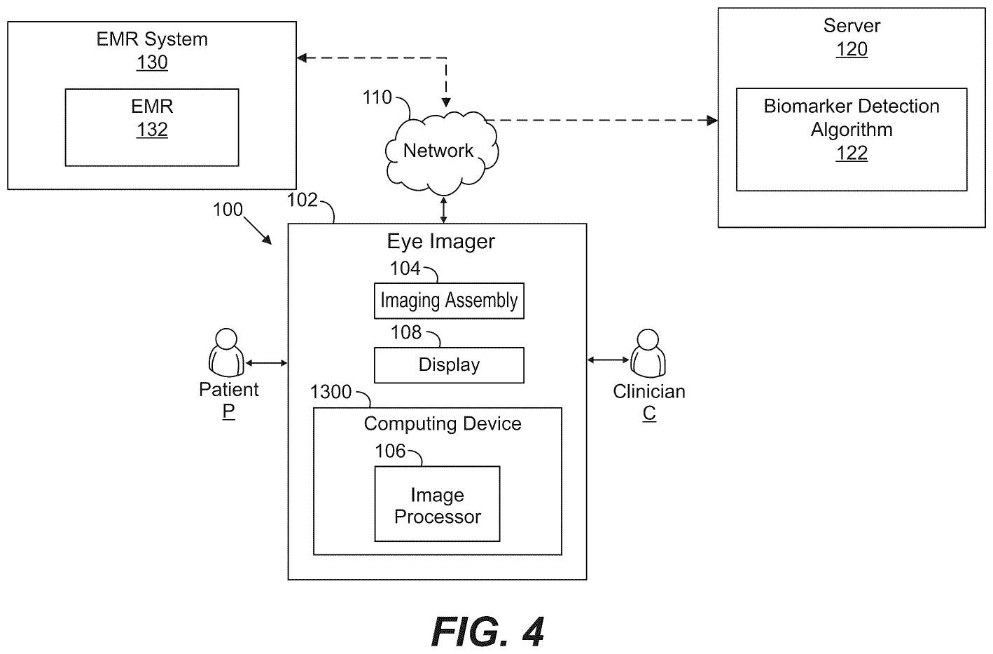

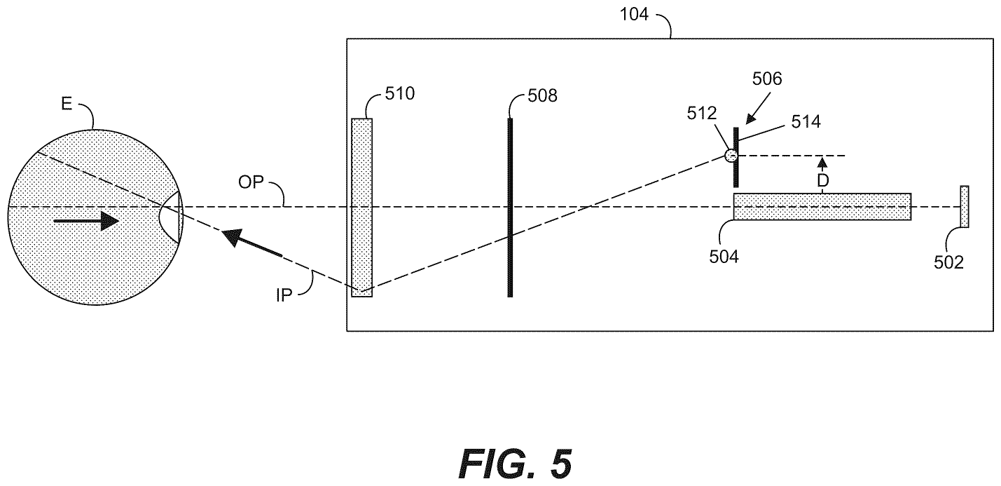

The second end 114 of the housing 102 includes a surface 116 that is shaped and sized for engaging the patient P's face. For example, the surface 116 can be positioned again the patient P's face such that the surface 116 surrounds both eyes of the patient P, as shown in FIG. 3. The second end 114 further includes a cavity 118. An imaging assembly 104 (see FIGS. 4 and 5) is configured to move along at least three axes of movement such as an x-axis (e.g., between left and right sides), a y-axis (e.g., between up and down), and a z-axis (e.g., between forward and rearward) relative to the patient P's face to image both the left and right eyes of the patient P while the housing 102 is held against the patient P's face.

FIG. 4 schematically illustrates an example of the eye imager 100. The eye imager 100 is operable by a clinician C to capture and view the one or more retinal images of the patient P. In some examples, the clinician C is an eyecare specialist such as an optometrist or an ophthalmologist. Alternatively, the clinician C can be a medical professional who is not trained as an eyecare specialist such as a general practitioner or primary care physician. In such examples, the eye imager 100 can be used to screen for both eye diseases and neurodegenerative diseases in a general practice medical office or other type of medical clinic.

As shown in FIG. 4, the eye imager 100 includes a computing device 1300 having an image processor 106. The eye imager 100 further includes an imaging assembly 104 in communication with the computing device 1300, and the display 108 in communication with the computing device 1300. The imaging assembly 104 captures digital retinal images of the patient P, and the display 108 displays the captured retinal images for viewing by the clinician C.

The imaging assembly 104 includes a lens, an aperture, and an image sensor, which are described in more detail with reference to FIG. 5. The imaging assembly 104 is configured to capture retinal images one eye at a time. In other examples, the imaging assembly 104 can capture retinal images of both eyes substantially simultaneously. In such examples, the eye imager 100 can include two separate imaging assemblies, one for each eye.

In the example shown in FIGS. 1-4, the display 108 is supported by the housing 102. In other examples, the display 108 is external of the eye imager 100, such as on a separate smartphone, tablet computer, or external monitor. The display 108 functions to display the images produced by the imaging assembly 104 in a size and format for viewing by the clinician C. In some examples, the display 108 is a liquid crystal display (LCD) or active matrix organic light emitting diode (AMOLED) display. In some examples, the display 108 is touch sensitive.

The eye imager 100 is connected to a network 110. The eye imager 100 can upload the retinal images captured by the imaging assembly 104 to a server 120 via the network 110. In at least some examples, the server 120 is a cloud server or other type of remote server.

In some examples, the eye imager 100 is operated by the clinician C to capture one or more retinal images of the patient P's eyes, the eye imager 100 transfers the retinal images via the network 110 to the server 120 for analysis by a biomarker detection algorithm 122, and the eye imager 100 receives via the network 110 a recommendation and/or diagnosis from the server 120. The biomarker detection algorithm 122 can be trained by artificial intelligence or machine learning to identify a presence in the retinal images of biomarkers associated with a neurodegenerative disease such as Alzheimer's disease. For example, the biomarker detection algorithm 122 can distinguish biomarkers associated with a neurodegenerative disease from other types of biomarkers or features that may be present in the retinal images.

In alternative examples, the biomarker detection algorithm 122 can be stored on a memory of the computing device 1300 of the eye imager 100 such that analysis of the retinal images for detection of biomarkers associated with neurodegenerative diseases is performed locally on the eye imager 100, and a recommendation and/or diagnosis relative to a risk or likelihood of developing a neurodegenerative disease is generated locally on the eye imager 100.

In some examples, the eye imager 100 and/or the server 120 are connected to an electronic medical record (EMR) (alternatively termed electronic health record (EHR)) system 130. The eye imager 100 and/or the server 120 can store the retinal images of the patient P, the detection of the biomarkers in the retinal images, and/or the recommendation and/or diagnosis in an electronic medical record (EMR) 132 of the patient P in the EMR system 130.

In examples where the clinician C is not a specialist, such as when the eye imager 100 is used for screening for eye diseases and neurodegenerative diseases in a general practice medical office, a retail clinic, or in the patient P's home, the retinal images, the detection of the biomarkers in the retinal images, and/or the recommendation and/or diagnosis stored in the EMR 132 of the patient P can be accessed by an overread clinician who is a specialist such as an ophthalmologist or neurologist. Thus, the retinal images, the detection of the biomarkers in the retinal images, and/or the recommendation and/or diagnosis can be accessed and viewed on another device by a remotely located clinician. Thus, the clinician who operates the eye imager 100 can be different from the clinician who evaluates the retinal images.

The network 110 may include any type of wireless network, wired network, or any combination of wireless and wired networks. Wireless connections can include cellular network connections and Wi-Fi. In some examples, a wireless connection can be accomplished directly between the eye imager 100 and an external display device using one or more wired or wireless protocols, such as Wi-Fi, Bluetooth, and the like. Other configurations are possible.

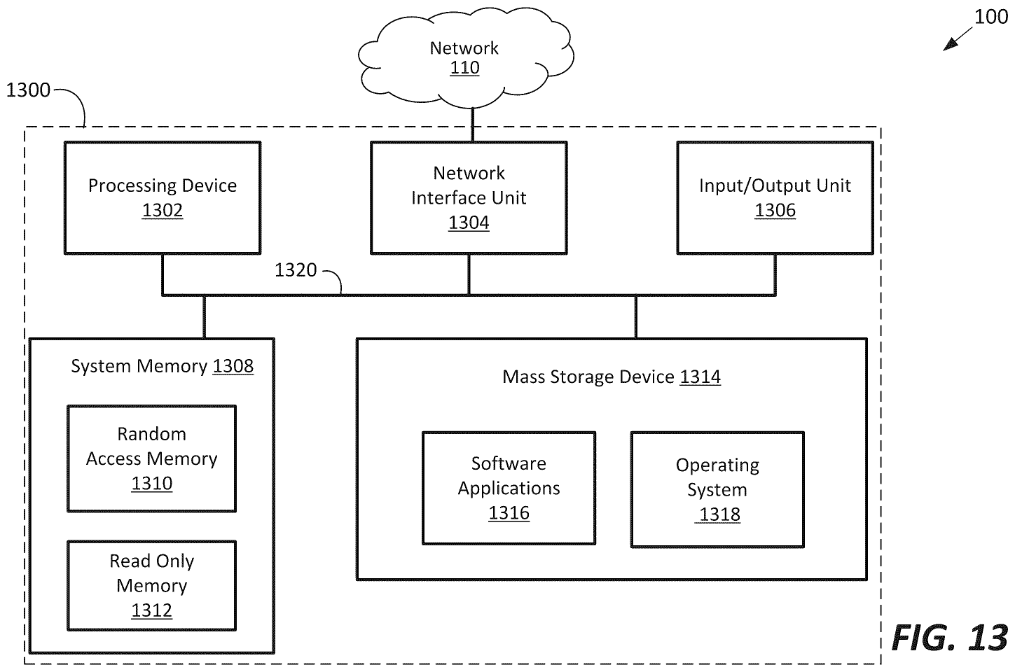

The image processor 106 is coupled to the imaging assembly 104 and is configured to communicate with the network 110 and the display 108. The image processor 106 can regulate the operation of the imaging assembly 104. An example of the computing device 1300 is shown in more detail in FIG. 13, which will be described further below.

FIG. 5 is a side view schematically illustrating an example of the imaging assembly 104 positioned relative to an eye E of the patient P such as during image capture by the eye imager 100. In the example shown in FIG. 5, the imaging assembly 104 includes an image sensor 502, a lens unit 504, an illumination unit 506, an aperture 508, and a front lens 510. The imaging assembly 104 can include additional or fewer components than those shown in FIG. 5.

The image sensor 502 captures light reflection from the eye E along an optical path OP and converts the light reflection into electrical signals to form a retinal image of the eye E. In some examples, the image sensor 502 is a digital image sensor such as an active-pixel sensor (APS) that uses a complementary metal oxide semiconductor (CMOS).

The lens unit 504 can include a plurality of lenses, filters, and additional elements that focus and refine the light reflection from the eye E toward the image sensor 502. The lens unit 504 at least partially defines the optical path OP of the imaging assembly 104.

The aperture 508 is an opening through which the light reflection from the eye E travels along the optical path OP. The aperture 508 is controlled by the image processor 106 to control the amount of light that passes through the lens unit 504 to the image sensor 502. The front lens 510 focuses the light reflection from the eye E into the imaging assembly 104.

The illumination unit 506 is in communication with the computing device 1300, which can coordinate the operation of the illumination unit 506 with adjustments of the aperture 508 and the lens unit 504 for capturing retinal images of the eye E of the patient P.

The illumination unit 506 includes one or more light-emitting diodes (LEDs) 512 which emit visible light such as white light in an illumination path IP toward the eye E. The visible light is used to illuminate the eye E for capturing the retinal images. As shown in FIG. 5, the one or more LEDs 512 are mounted on a printed circuit board 514.

The illumination unit 506 can also include one or more near-infrared LEDs that generate near-infrared illumination during a preview mode of the eye imager 100. For example, the illumination unit 506 is an assembly including one or more visible light LEDs and one or more near-infrared LEDs. The one or more near-infrared LEDs are used in the preview mode to allow the eye imager 100 to determine or estimate a location of the pupil of the eye E without illuminating the eye E with visible light that could cause the pupil to contract or irritate the patient P. Once the location of the pupil of the eye E is identified, the computing device 1300 positions the imaging assembly 104 relative to the eye E, and controls the one or more visible light LEDs to illuminate the eye E with visible light for capturing one or more retinal images.

The illumination unit 506 is mounted relative to the lens unit 504 such that the one or more LEDs 512 are offset with respect to the lens unit 504 causing the illumination path IP to be offset with respect to the optical path OP. In the example embodiment shown in FIG. 5, the illumination unit 506 is mounted above the lens unit 504. In alternative example embodiments, the illumination unit 506 can be mounted below the lens unit 504.

As shown in FIG. 5, the one or more LEDs 512 are offset with respect to the lens unit 504 by a distance D. As an illustrative example, the distance D is about 12 millimeters (mm). The distance D causes the illumination path IP to be offset with respect to the optical path OP.

In the example shown in FIG. 5, a distance between the one or more LEDs 512 and the front lens 510 produces a focal point on a side of the front lens 510 opposite of the one or more LEDs 512. This causes the illumination path IP to refract towards a center of the optical path OP. In such examples, the front lens 510 is a converging lens.

As will now be described in more detail, the offset between the illumination path IP and the optical path OP causes the retinal images capture by the imaging assembly 104 to more clearly depict biomarkers that are associated with one or more neurodegenerative diseases. For example, the offset between the illumination path IP and the optical path OP causes the biomarkers to appear in the retinal images captured by the imaging assembly 104 without requiring any additional post-processing for identifying the biomarkers in the retinal images. Additionally, the biomarkers appear in the retinal images captured by the imaging assembly 104 without requiring mydriasis, which can be painful and/or inconvenient to the patient P.

In one illustrative example, the biomarkers include amyloid plaque that is associated with a likelihood of developing or having Alzheimer's disease. Amyloid plaque (also known as neuritic plaque, amyloid beta plaque or senile plaque) is an extracellular deposit of the amyloid beta (AB) protein. Amyloid plaque typically occurs in the brain, but may also be found in the retina of the eye. Amyloid plaque is typically produced by degenerative neuronal elements such that amyloid plaque is a characteristic feature of Alzheimer's disease.

Alzheimer's disease usually starts slowly and progressively worsens. In some instances, amyloid plaque is present in the retina of the eye E before other symptoms of Alzheimer's disease materialize such as memory loss, speech impairment, disorientation, mood swings, and other symptoms. By identifying presence of amyloid plaque in the retinal images captured by the eye imager 100, a likelihood or risk of developing Alzheimer's disease can be detected earlier such that medications that delay progression of Alzheimer's disease can be prescribed sooner to improve the patient P's quality of life and life expectancy. As an illustrative example, amyloid plaque can be detected in the retina 10 to 20 years before the onset of Alzheimer's symptoms allowing medications to significantly delay progression of the disease.

The offset between the illumination path IP and the optical path OP causes the retinal images capture by the imaging assembly 104 to more clearly depict biomarkers such as amyloid plaque because the biomarkers are illuminated at an angle relative to the optical path OP, rather than being illuminated head-on, which occurs when there is on-axis illumination of the eye E (i.e., when the illumination unit 506 is not offset by the distance D with respect to the lens unit 504). The off-axis illumination causes a brighter and clearer refraction from the biomarkers.

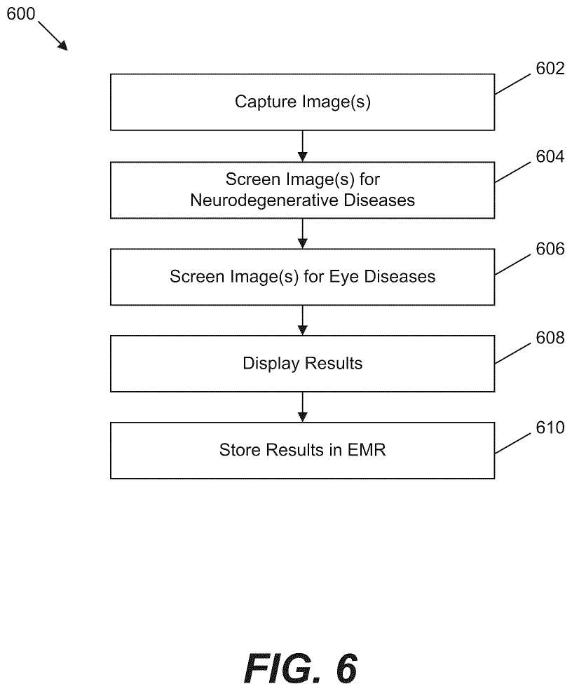

FIG. 6 schematically illustrates an example of a method 600 of performing a retinal exam of the patient P. The method 600 can be performed by the eye imager 100. The method 600 includes an operation 602 of capturing one or more retinal images. For example, operation 602 can include capture one or more retinal images of the left eye and one or more retinal images of the right eye of the patient P. As discussed above with respect to FIG. 3, the imaging assembly 104 is controlled by the computing device 1300 to move along at least three axes of movement such as an x-axis (e.g., left and right), a y-axis (e.g., up and down), and a z-axis (e.g., forward and rearward) relative to the patient P's face to image both the left and right eyes of the patient P while the housing 102 is held against the patient P's face.

The method 600 includes an operation 604 of screening the one or more retinal images captured in operation 602 for detection of biomarkers associated with neurodegenerative diseases. Operation 604 can include using the biomarker detection algorithm 122 (see FIG. 4) to analyze the one or more retinal images for detection of the biomarkers. In some examples, the biomarker detection algorithm 122 is performed on the server 120 such that the eye imager 100 sends via the network 110 the retinal images to the server 120 for analysis, and the eye imager 100 receives via the network 110 the results of the analysis from the server 120. In other examples, the biomarker detection algorithm 122 is performed locally on the eye imager 100.

The biomarker detection algorithm 122 is trained by machine learning to identify a presence in the retinal images of biomarkers associated with a neurodegenerative disease such as Alzheimer's disease. For example, the biomarker detection algorithm 122 can distinguish biomarkers associated with a neurodegenerative disease from other types of biomarkers or artifacts that may be present in the retinal images. As an illustrative example, the biomarker detection algorithm 122 can be trained to identify amyloid plaque in the retinal images such that the biomarker detection algorithm 122 distinguishes amyloid plaque from other types of biomarkers such as exudates and drusen. For example, the biomarker detection algorithm 122 can be trained to learn the shape, size, coloring, and other characteristics of amyloid plaque to distinguish the amyloid plaque from other types of biomarkers.

As a further illustrative example, the biomarker detection algorithm 122 identifies implicit visual features correlating to neurodegenerative diseases apart from amyloid plaque. Such implicit visual features may not be directly observable or describable by human experts, but can be derived through machine learning. The biomarker detection algorithm 122 learns through machine learning to extract such implicit features to provide complementary evidence when searching for presence of amyloid plaque in an area of interest in a digital retinal image.

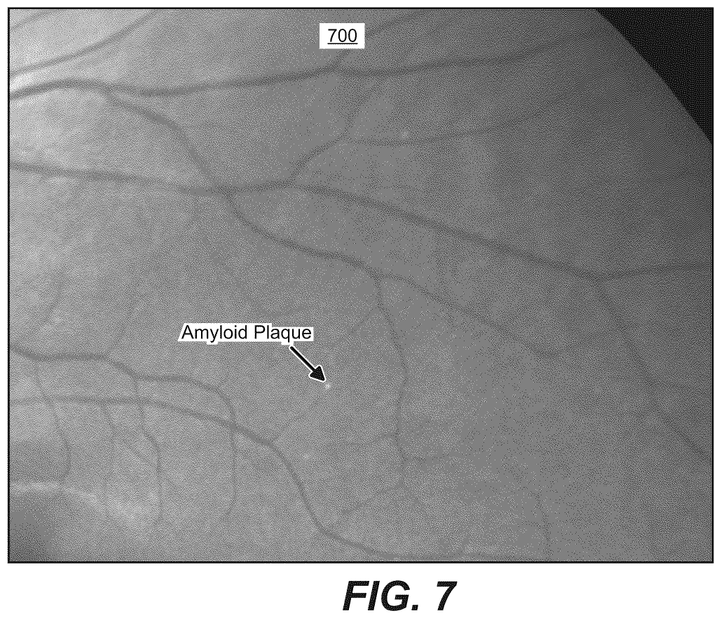

FIG. 7 is an example of a retinal image 700 captured by the eye imager 100. The retinal image 700 includes biomarkers associated with a neurodegenerative disease. In the example shown in FIG. 7, the retinal image 700 includes an amyloid plaque which appears as bright a spot on the retina due to the offset between the illumination path IP and the optical path OP of the imaging assembly 204 (see FIG. 5). As discussed above, no post processing of the retinal image 700 is performed for identifying the amyloid plaque. Also, the amyloid plaque appears in the retinal image 700 without mydriasis, which can be painful and/or inconvenient.

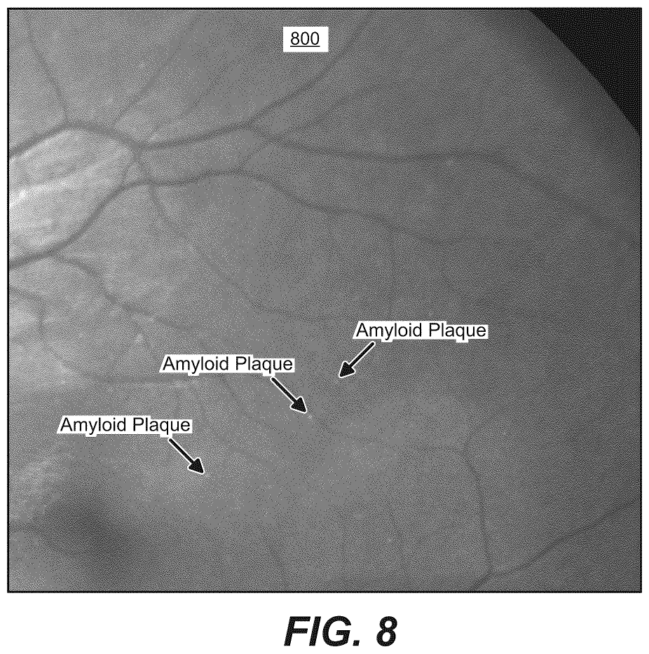

FIG. 8 is another example of a retinal image 800 captured by the eye imager 100. In the example of FIG. 8, the retinal image 800 includes amyloid plaque which appears as bright spots on the retina due to the offset between the illumination path IP and the optical path OP.

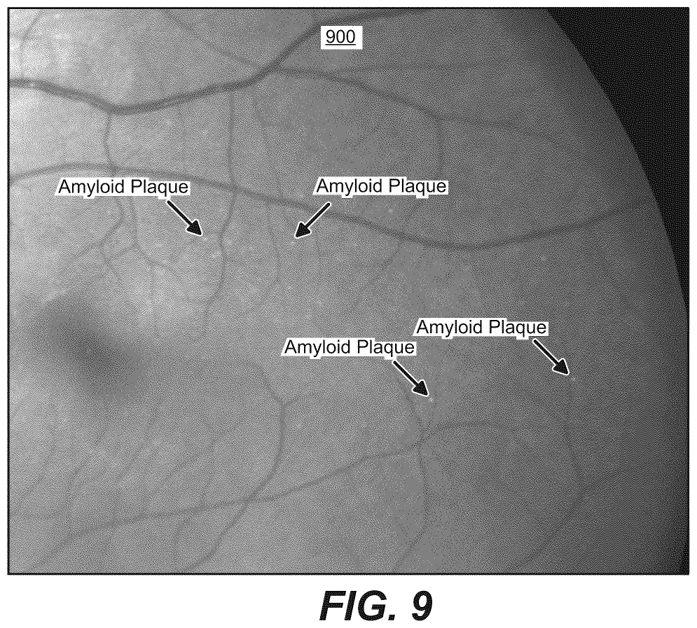

FIG. 9 is another example of a retinal image 900 captured by the eye imager 100. In the example of FIG. 9, the retinal image 900 includes amyloid plaque which appears as bright spots on the retina due to the offset between the illumination path IP and the optical path OP.

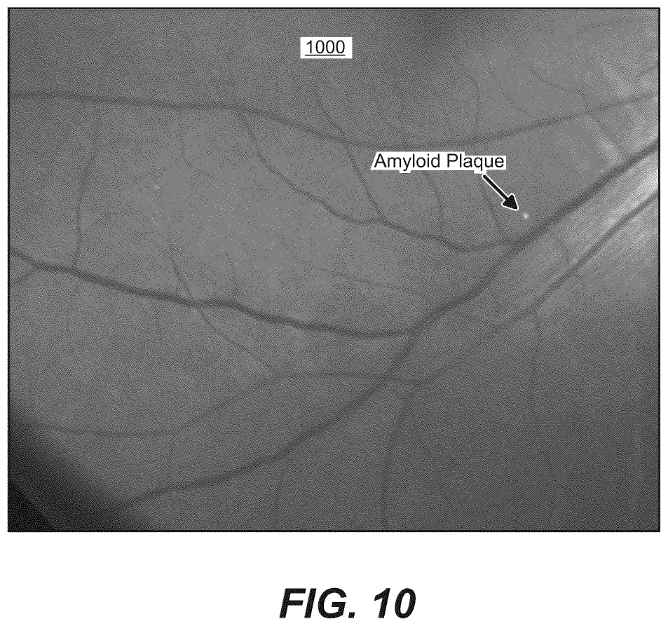

FIG. 10 is another example of a retinal image 1000 captured by the eye imager 100. In the example of FIG. 10, the retinal image 1000 has amyloid plaque which appears as bright spots on the retina due to the offset between the illumination path IP and the optical path OP.

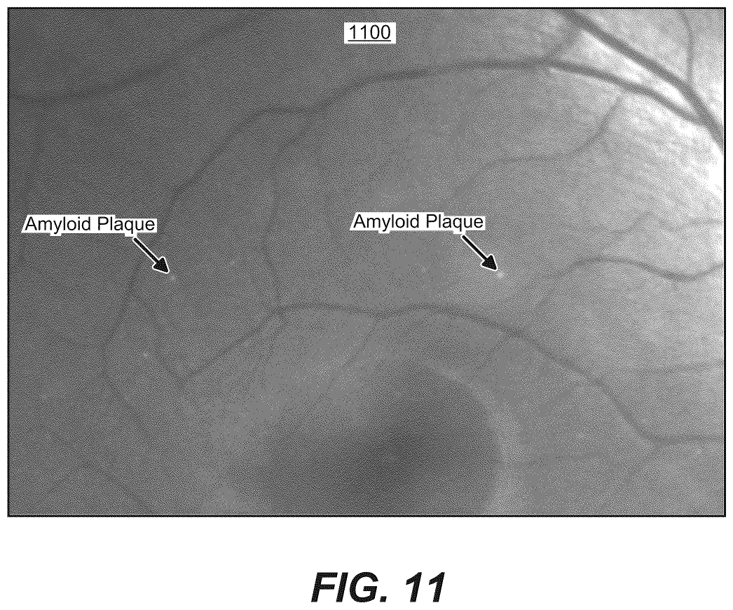

FIG. 11 is another example of a retinal image 1100 captured by the eye imager 100. In the example of FIG. 11, the retinal image 1100 has amyloid plaque which appears as bright spots on the retina due to the offset between the illumination path IP and the optical path OP.

Referring back to FIG. 6, the method 600 can include an operation 606 of screening the one or more retinal images captured in operation 602 for detection of one or more conditions associated with one or more eye diseases. For example, operation 606 can include screening the one or more retina images for conditions associated with at least one of diabetic retinopathy, macular degeneration, glaucoma, and papilledema. Advantageously, the retinal exam performed by the method 600 can screen for both neurodegenerative disease and eye diseases in one exam sitting using the same retinal images captured from the patient P. This provides a more comprehensive and efficient examination of the patient P because the patient P does not need to go through multiple types of exams that require use of different types of imagers. Instead, because of the unique off-axis illumination provided by the eye imager 100, the same retinal images can be screened for both neurodegenerative diseases and eye diseases in one sitting.

The method 600 further includes an operation 608 of displaying results from the screening of the one or more retinal images performed in operation 604 for the detection of biomarkers associated with neurodegenerative diseases. In examples where the biomarker detection algorithm 122 is performed on the server 120, the server 120 transmits via the network 110 the results to the eye imager 100 for display on the display 108. In examples where the biomarker detection algorithm 122 is performed locally on the eye imager 100, the results are displayed on the display 108 without communicating with the server 120 over the network 110.

The results displayed in operation 608 can include displaying whether the biomarker is detected or not. In examples where a biomarker such as amyloid plaque is detected, the results can include a risk or likelihood of having Alzheimer's disease. The results displayed in operation 608 can further include a referral to follow-up with a specialist such as a neurologist.

Operation 608 can further include displaying results from the screening of the one or more retinal images performed in operation 606 for the detection of one or more conditions associated with one or more eye diseases. For example, the results displayed in operation 608 can include a risk or likelihood of having one or more eye diseases such as diabetic retinopathy, macular degeneration, glaucoma, and papilledema. In such examples, results displayed in operation 608 can further include a referral to follow-up with another type of specialist such as an ophthalmologist. Thus, the results displayed in operation 608 can include recommendations regarding both neurodegenerative diseases and eye diseases from a single retinal examination.

In some examples, the method 600 includes an operation 610 of storing the one or more retinal images and the results in the EMR 132 of the patient P. Operation 610 allows the one or more retinal images and the results displayed in operation 608 to be accessible or otherwise made available to a specialist such as a neurologist and/or an ophthalmologist for further clinical analysis. Accordingly, the method 600 can be performed on the eye imager 100 when in a general medical practice setting such as a medical office of a primary care physician to screen for neurological and eye diseases, and depending on the results from the screening performed in operations 604, 606, a referral to a specialist such as a neurologist or ophthalmologist can be made. Thus, the method 600 enables earlier detection of a risk or likelihood of having a neurological disease or eye disease allowing for earlier treatment and mitigation of symptoms before permanent damage to the brain or eyes occurs.

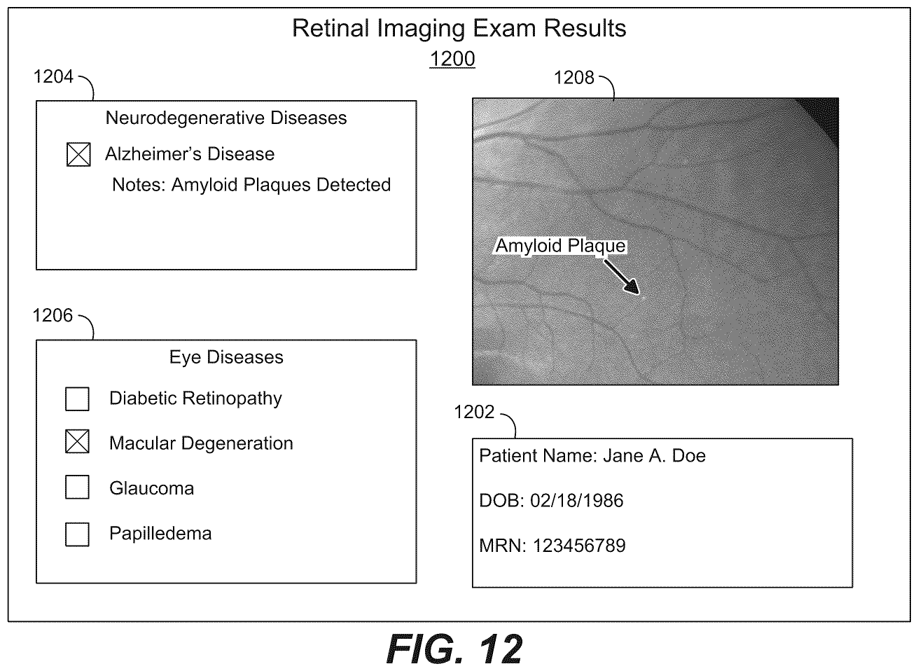

FIG. 12 illustrates an example of a graphical user interface 1200 displayed in accordance with an example of operation 608 of the method 600. As shown in this example, the graphical user interface 1200 includes patient information 1202 such as the patient P's name, date of birth, and medical record number. The graphical user interface 1200 includes neurodegenerative disease results 1204 from screening the one or more retinal images captured by the eye imager 100. In the example shown in FIG. 12, the neurodegenerative disease results 1204 indicate that there is a risk or likelihood of developing Alzheimer's disease. Additionally, a reason or an explanation for the results is displayed (e.g., “Amyloid plaque detected”).

The graphical user interface 1200 can further include a retinal image 1208 that shows detection of the biomarker on which the neurodegenerative disease results 1204 are based. In the example in FIG. 12, the retinal image 1208 shows an amyloid plaque which is indicative of a risk or likelihood of developing Alzheimer's disease.

The graphical user interface 1200 can further include eye disease results 1206 from screening the one or more retinal images captured by the eye imager 100. In the example shown in FIG. 12, the eye disease results 1206 indicate that there is a risk or likelihood of macular degeneration, but that other eye diseases such as diabetic retinopathy, glaucoma, and papilledema are not detected from the retinal images captured by the eye imager 100.

FIG. 13 schematically illustrates example components of the computing device 1300 of the eye imager 100. The server 120 and the EMR system 130 can include similar computing devices. As shown in FIG. 13, the computing device 1300 includes at least one processing device 1302, a system memory 1308, and a system bus 1320 coupling the system memory 1308 to the at least one processing device 1302. The at least one processing device 1302 is an example of a processor such as a central processing unit (CPU) or microcontroller.

The system memory 1308 is an example of a computer readable data storage device that stores software instructions that are executable by the at least one processing device 1302. The system memory 1308 includes a random-access memory (“RAM”) 1310 and a read-only memory (“ROM”) 1312. Input/output logic containing the routines to transfer data between elements within the eye imager 100, such as during startup, is stored in the ROM 1312.

The computing device 1300 can include a mass storage device 1314 that is able to store software instructions and data. The mass storage device 1314 can be connected to the at least one processing device 1302 through a mass storage controller connected to the system bus 1320. The mass storage device 1314 and associated computer-readable data storage medium provide non-volatile, non-transitory storage for the eye imager 100.

Although the description of computer-readable data storage media contained herein refers to a mass storage device, the computer-readable data storage media can be any non-transitory, physical device or article of manufacture from which the device can read data and/or instructions. The mass storage device 1314 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The eye imager 100 can operate in a networked environment through connections to remote network devices and systems connected to the network 110. The eye imager 100 connects to the network 110 through a network interface unit 1304 connected to the system bus 1320. The network interface unit 1304 can also connect to other types of networks and remote systems.

The eye imager 100 can also include an input/output controller 1306 for receiving and processing input from a number of input devices such as a touchscreen display. Similarly, the input/output controller 1306 may provide output to a number of output devices.

The mass storage device 1314 and the RAM 1310 can store software instructions and data. The software instructions can include an operating system 1318 suitable for controlling the operation of the eye imager 100. The mass storage device 1314 and/or the RAM 1310 also store software instructions 1316, that when executed by the at least one processing device 1302, cause the eye imager 100 to provide the functionalities discussed in this document.

The various embodiments described above are provided by way of illustration only and should not be construed to be limiting in any way. Various modifications can be made to the embodiments described above without departing from the true spirit and scope of the disclosure.

What is claimed is:

1. A device for neurodegenerative disease screening, the devise comprising:
an imaging assembly including:
an image sensor for capturing a retinal image;
a lens unit focusing light reflection from an eye toward the image sensor, the lens unit at least partially defining an optical path for receiving the light reflection; and
an illumination unit having one or more light-emitting diodes emitting light in an illumination path for illuminating the eye, the illumination unit being mounted relative to the lens unit such that the illumination path is offset with respect to the optical path;
at least one processing device; and
at least one computer readable data storage device storing software instructions that, when executed by the at least one processing device, cause the device to:
operate the imaging assembly to capture the retinal image using the illumination path offset with respect to the optical path; and
receive a recommendation based on detection of a biomarker in the retinal image, the biomarker being associated with a neurodegenerative disease.

2. The device of claim 1, wherein the biomarker is amyloid plaque, and the recommendation includes identification of a risk for developing Alzheimer's disease.

3. The device of claim 2, wherein the recommendation further includes a referral for following up with a neurologist.

4. The device of claim 1, wherein the instructions, when executed by the at least one processing device, further cause the device to:
receive a detection of one or more conditions in the retinal image associated with eye diseases including at least one of retinopathy, macular degeneration, glaucoma, and papilledema.

5. The device of claim 4, wherein the recommendation further includes a referral for following up with an ophthalmologist.

6. The device of claim 1, wherein the biomarker is detected by an algorithm trained by machine learning.

7. The device of claim 1, wherein the retinal image is captured using a non-mydriatic workflow.

8. The device of claim 1, wherein the instructions, when executed by the at least one processing device, further cause the device to:
store the retinal image and the recommendation in an electronic medical record that is accessible by a specialist for further clinical assessment.

9. A method of screening for a neurodegenerative disease, the method comprising:
capturing a retinal image by operating an imaging assembly having an illumination path that is offset with respect to an optical path;
detecting a biomarker in the retinal image, the biomarker being associated with the neurodegenerative disease; and
generating a recommendation based on detection of the biomarker.

10. The method of claim 9, wherein the biomarker is amyloid plaque, and the recommendation includes identification of a risk for developing Alzheimer's disease.

11. The method of claim 10, wherein the recommendation further includes a referral for following up with a neurologist.

12. The method of claim 9, further comprising:
screening the retinal image for one or more conditions associated with eye diseases including at least one of retinopathy, macular degeneration, glaucoma, and papilledema.

13. The method of claim 12, wherein the recommendation further includes a referral for following up with an ophthalmologist.

14. The method of claim 9, wherein the biomarker is detected by an algorithm trained by machine learning.

15. The method of claim 9, wherein the retinal image is captured using a non-mydriatic workflow.

16. The method of claim 9, further comprising:
storing the retinal image and the recommendation in an electronic medical record accessible by a specialist for further clinical assessment.

17. A system for neurodegenerative disease screening, the system comprising
at least one processing device; and
at least one computer readable data storage device storing software instructions that, when executed by the at least one processing device, cause the device to:
receive a retinal image captured using a non-mydriatic workflow;
screen the retinal image for amyloid plaque associated with Alzheimer's disease;
screen the retinal image for conditions associated with eye diseases; and
generate a recommendation, wherein the recommendation includes a referral to a neurologists when the amyloid plaque is detected in the retinal image, and wherein the recommendation includes a referral to an ophthalmologist when the conditions associated with the eye diseases are detected in the retinal image.

18. The system of claim 17, wherein the eye diseases include at least one of retinopathy, macular degeneration, glaucoma, and papilledema.

19. The system of claim 17, wherein the amyloid plaque is detected by an algorithm trained by machine learning.

20. The system of claim 17, wherein the instructions, when executed by the at least one processing device, further cause the device to:
store the retinal image and the recommendation in an electronic medical record that is accessible by a specialist for further clinical assessment.
